(19) United States
(12) Patent Application Publication
(10) Pub. No.: US 2008/0302369 A1
(43) Pub. Date: Dec. 11, 2008

(54) Intra-Vas Deferens Occlusive Device for Male Contraception

(75) Inventor: Aurelio Uson Calvo, Madrid (ES)

Correspondence Address: RICHARD M. GOLDBERG, 25 EAST SALEM STREET, SUITE 419, HACKENSACK, NJ, 07601, US

(73) Assignee: INVENCIONES MEDICO SANITARIAS S.L., Madrid (ES)

(21) Appl. No.: 11/571,858

(22) PCT Filed: Jul. 14, 2005
(86) PCT No.: PCT/ES2005/000399

§ 371 Date: Jan. 9, 2007

(30) Foreign Application Priority Data
Jul. 16, 2004 (ES) P200401760

(51) Int. Cl. A61F 6/02

(52) U.S. Cl. 128/843

(57) ABSTRACT

An intra-vas deferens occlusive device for male contraception includes an axial filament having a small identification ball at the proximal end thereof, which remains outside the vas deferens but which is joined to an anchoring filament and to two occlusive bodies essentially in the form a clamp. The occlusive bodies are joined in a first embodiment by the interconnecting filament and in a second embodiment by the vertices thereof, such as to form a peripheral slit. Both embodiments terminate in a flange which is assembled to a washer pivot of the ad hoc insertion and implantation needle of the device.

### PURPOSE OF THE INVENTION

The present descriptive report refers to an invention patent application corresponding to an improved intra-vas deferens occlusive device, the main purpose of which is to produce permanent total male infertility after correct intra-duct implantation, by means of a simple surgical technique, which is only minimally invasive, carried out under local anaesthesia and on an outpatient basis.

This device incorporates a double obstructive barrier that totally impedes and blocks the passage of sperm to the outside just at the intra-lumen zone of both vas deferens ducts where said obstructive bodies are located, leaving the seminal ejaculate free from sperm and hence, the user sterile.

In addition, because of the small size, easy identification and anatomical localisation by digital palpation, after intra-duct implantation, it is also very easy to subsequently extract it under local anaesthesia and on an outpatient basis, when the user wishes to recover his interrupted fertility because the reversibility of the procedure is more easily achieved at a much lower cost and with improved results than those obtained in men who have undergone a vasectomy that are subjected to the complicated, costly micro-surgical technique, known as vasectomy reversal, if they wish to recover their interrupted fertility.

### SCOPE OF THE INVENTION

The main application of this invention is medical praxis, especially in minor surgery services or units for outpatients, where the implantation and extraction of this device is generally practiced for permanent or temporary control of male fertility. This invention also has application scope within the industry that manufactures health complements, accessories and elements for internal use.

### BACKGROUND OF THE INVENTION

In their sexual relations, men currently make use of various contraceptive methods, such as sexual abstinence, “coitus interruptus”, the use of condoms and/or bilateral vasectomy. The invention adds a new much less aggressive double-barrier technique when compared to the contraceptive methods currently employed by men that live in the so-called technified or developed countries, which is also much more comfortable and safer than a condom and without the possible complications and/or sequelae resulting from a bilateral vasectomy.

In this respect, better information and constancy must be available about the problems involved in the inappropriate use of condoms and also complications and/or sequelae resulting from a standard bilateral vasectomy.

In the case of the condom employed exclusively as an anticonceptive method, it does not provide one hundred percent efficiency and does not provide total safety and reliability to the user, so that it cannot guarantee total, permanent infertility. The use of a condom also leads to evident reduction in sexual pleasure for male/female couples during vaginal coitus, together with a certain discomfort or trouble to the male when putting it on or taking it off, and the fear that it will tear during vaginal coitus. Lastly, there is the high economic cost involved in long-term use of condoms when continually and exclusively employed as an anticonceptive method.

There are many men who have undergone vasectomies that suffer from post-operation complications and sequelae because this intervention involves the mutilation of both vas deferens ducts and hence, there are those who suffer a psychological feeling of rejection for this technique and even of castration or erectile dysfunction or lack of libido (sexual appetite).

To all this must be added the frequent irreversibility of the standard bilateral vasectomy that depends on many factors, among which are the following:

1. The time elapsed since the contraceptive bilateral vasectomy and the vas deferens point or location where said surgery was performed.

2. The existence or not of post vasectomy epididymis lesions, such as micro-ruptures with or without spermiocyte granulomas in one or more sites of the very fine, intricate epididymis duct. Also the existence of generalised atrophy of the tumulus germinal tissue.

3. Consideration must also be given to possible intra and/or post-operation complications deriving from the use of general or local anaesthesia and if this is not enough, there is also the complexity and the high cost of the surgical intervention, any possible complications and, of course, the unpredictability of post-operation results.

All the previously indicated problems about the drawbacks, difficulties and possible complications or sequelae involved in both the indiscriminate use of condoms and the practice of the standard bilateral vasectomy can be easily resolved through the technique of intra-duct implantation of the above-mentioned occlusive infra-deferens device as proposed by the invention.

It should also be stated that the inventor is the owner of an occlusive infra-deferens device with a double-barrier for male contraception that was registered at the Spanish Patent and Trademark Office on the 8 Mar. 1989 under the name of Aurelia Usón Calvo and also that said invention is currently obsolete. For this reason, the following changes and/or modifications are cited, among others:

1. The device has been reduced in size and the structure ergonomically improved.

2. The shape and position of some components have been modified.

3. The identification ball has been moved to one of its ends.

4. The other end bears a flange to which an additional element, called the intra-duct insertion needle for the occlusive infra-deferens device is joined, which will be described in fuller detail later.

### DISCLOSURE OF THE INVENTION

As has been pointed out, the basic purpose of the new, improved occlusive infra-deferens device for male contraception as proposed by the invention is to produce permanent total infertility of the user and also achieve this in a much simpler fashion than a bilateral vasectomy, in other words, without any significant damage to the deferens ducts, allowing subsequent reversibility by simply extracting the device under local anaesthesia on an outpatient basis.

The improved occlusive infra-deferens device comprises an axial filament on which a spherical body (1) is mounted at one end and to which the anchoring filament (2) is attached that goes from the cited spherical body to the proximal vertex of the first occlusive barrier (3), generally in the form of an elongated barrel, although it could have any other shape, configuration and/or size. There is a short axial filament, now called the interconnection filament (4) attached to this first barrier, which is connected to the proximal vertex of the second occlusive barrier (5) producing identical configuration and dimensions to the first, although this also, could be different. The invention finally incorporates a flange (6) that emerges at the protrudes by the distal vertex of the second barrier that is axially connected to the pivot with washers (7) that centre of the proximal end of the insertion needle (8), with the contraceptive device firmly secured to its insertion needle during the manufacturing process, and therefore joined together to form a single piece as will be described later.

The invention is currently represented by two models or versions of the improved infra-deferens occlusive device, which are:

1. The occlusive device fitted with two separated barriers, known as the “Twin-plug” or model “A” of the invention.

2. The occlusive device fitted with two barriers joined together by a perimeter cleft located in the centre of the obturator body and known as the model “B” or “double single-plug” (see attached drawing).

The second version of the invention, the model “B”, with the double single-plug, does not have the interconnection filaments and, at the central part of this obstructive block, there is a perimeter cleft (FIG. 2 of the drawing), with the rest of the component parts of this device identical to those described for model “A” or “twin-plug”.

The name and function or purpose of each element that structurally makes up the improved device in the cited models are as follows: 1) the spherical body incorporated at the distal end of the anchoring filament is known as the ball, which remains outside the vas deferens duct after intra-duct implantation of the rest of the device, is used to identify its anatomical position inside the user via digital palpation of the cited sphere, which is located under the skin and scrotal wall that covers it.

The so-called anchoring filament that crosses the deferens duct wall is joined by one end of the spherical body, which is outside the vas deferens duct and by the other to the proximal vertex of the first barrier, with a large part of this filament inside said duct and which, as the name indicates, fixes or anchors the rest of the contraceptive device in a stable intra-duct position.

The so-called occlusive bodies, with their elongated barrel shapes, of the “twin-plug” or model “A” device are separated and, in turn, joined by the interconnection filament. On the other hand, the obstructive block, with its elongated barrel shape truncated at one of its vertices and joined together by a perimeter cleft makes up the Model “B” or “double single-plug”. Both the “A” and “B” models are used to obstruct the span of the vas deferens ducts, thus completely blocking the passage of sperm right in the zone where said barriers are implanted. On the other hand, the so-called joining or interconnection filament located between the two barriers in the “A” model or twin-plug device is used to maintain the two barriers a certain distance apart. The flange or terminal component in the two models proposed by the invention, is configured by the axial filament that extends by the distal vertex of the second barrier, which is axially assembled to the insertion needle pivot with washers that is identical in both models “A” and “B” of the intra-duct occlusive device. FIGS. 4 and 5 on the drawing. Consequently, the contraceptive device proposed by the invention in its two versions or models, are jointly and simultaneously manufactured with medical-grade silicone, with its corresponding insertion needle, which is placed into the mould just after the flange, thus obtaining a complete occlusive or “assembled” or “dressed” device of models “A” and “B”, ready for marketing and correct trans-parietal and intra-duct implantation in both vas deferens ducts. Naturally, among other legal requirements, the latter is preceded by:

1. Packaging and commercial labelling.

2. Its date of packaging and sterilisation, together with its expiry dates.

3. Date of official, legal approval, explanatory leaflet, device details and, of course, the corresponding informed, signed patient's consent.

In addition, the insertion needle for both models of the intra-vas deferens contraceptive device is specially designed for longitudinal assembly opposite the point of its respective flanges on the two cited models currently proposed by the invention. In fact, the insertion needle for the intra-vas deferens occlusive device is the male option. The proximal end of this needle has a fine shaft or pivot with several washers around it in order for the liquid silicone that is injected in the corresponding manufacturing mould in each of the two device models completely covers said central shaft or pivot of the needle, including its washers and also part of the distal half of the flange on both models, so that both components, the intra-vas deferens contraceptive device and the insertion needle configure a single body, thus facilitating quick, correct intra-duct implantation. Once device implantation is achieved, the needle is discarded after transversal cutting of its connection with the device flange. It should also be pointed out the round configuration of said insertion needle point so that it does not scratch or tear the mucous membrane surrounding the span of the vas deferens ducts during the trans-parietal insertion process and intra-luminal implantation. In addition, as already mentioned, the proximal end of the insertion needle is thickened and attains a size and configuration very similar to that of the occlusive barriers or bodies in order to thus facilitate correct trans-parietal insertion and intra-luminal implantation and, of course, minimum possible aggression or damage in the vas deferens duct.

### DESCRIPTION OF THE DRAWINGS

As a complement to this description and in order to assist in a better understanding of the invention's characteristics, this descriptive report is accompanied by a figure drawing as an integral part of the same, which has a merely illustrative, non-limiting character, in which the following is represented:

FIG. 1 is a side elevation of a first exemplary embodiment of the model “A” of the twin-plug device, in relation to an improved intra-vas deferens occlusive device that is ideal for male contraception.

FIG. 2 is a side elevation view of the second exemplary embodiment of the invention or model “B” of the improved occlusive “double single-plug” intra-vas deferens device which, like model “A” shown in FIG. 1 incorporates the identification ball, anchoring filament and the flange, but with a single obstructive block or “double single-plug” in the form of an elongated barrel with a perimeter cleft at the connection point of its vertices of both elongated barrels. Model “B” is 20% shorter than model “A”.

FIG. 3 shows a side elevation view of the insertion needle, male option, longitudinally sectioned by its mid plane, used for the ad hoc implantation of the models of the two occlusive intra-vas deferens devices that the invention proposes as the most suitable male contraceptive means whether it is model “A” or “twin-plug” or the model “B” or double single-plug device.

FIG. 4 is a side elevation view that is longitudinally sectioned through its mid plane of the distal vertex of the second barrier in the shape of an elongated barrel or occlusive body, followed by the flange, the point of which faces the distal end of the pivot with washers that protrudes from the centre of the proximal end of the insertion needle, before this area of the joint is injected and sealed with medical grade liquid silicone. It also shows the rest of the insertion needle that terminates in a round point.

FIG. 5 shows a side elevation view that is longitudinally sectioned through its mid plane on a larger scale than that of FIG. 4, mainly of the assembly between the silicone-covered distal third of the flange and pivot with washers of the insertion needle forming a thickening that identical in size and configuration to the occlusive barriers, which facilitates the trans-parietal and intra-duct implantation of the of the contraceptive device.

FIG. 6 is a side elevation view of the model “A” or twin-plug device that is longitudinally sectioned through its mid plane and assembled with its insertion and intra-vas deferens needle, showing the connection between the distal third of the silicone-covered flange and pivot of the insertion needle.

FIG. 7 shows a side elevation view of the model “B” or double single-plug device that is longitudinally sectioned through its mid plane and assembled with its insertion needle. Note that the join or seal between the distal third of the flange and the pivot with washers of the insertion needle is identical to that of FIG. 6.

### EXEMPLARY EMBODIMENTS OF THE INVENTION

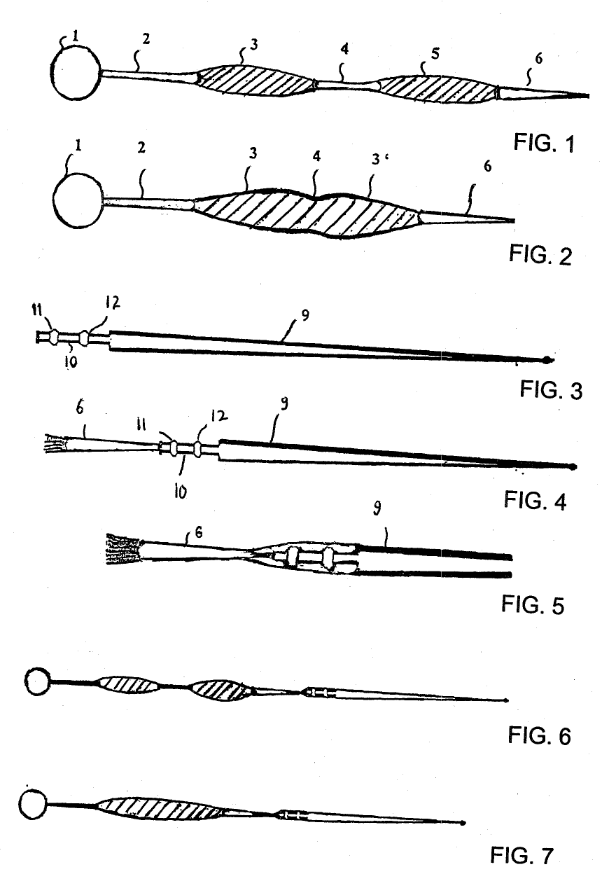

FIG. 1 shows a first embodiment of the occlusive model “A” or “twin-plug” intra-deferens device for male contraception. This device comprises an axial filament that runs from one end to the other of the whole device with the so-called identification and/or device localisation ball (1) at the proximal end and the so-called anchoring filament (2). After the anchoring filament comes two zones (3) and (5) for the two barriers, which, in this case are elongated barrel shaped, the proximal (3) and the distal (5) respectively, joined together by the interconnection filament (4), which ends in the proximal vertex of the second barrier (5), the distal vertex of which produces the axial filament now converted into the flange (6), which is axially assembled with the pivot with washers. See FIG. 4 located at the proximal end of the needle. These two elements, from the distal third of the flange, including the entire pivot with washers up to the proximal end of the needle are firmly joined together by the solidification of the liquid silicone injected during the manufacturing process of models “A” and “B” of the improved occlusive intra-vas deferens device proposed by the invention. See FIG. 5.

FIG. 2 is a second embodiment of the invention, model “B”, the so-called “double single-plug”, comprising the following elements: a spherical body of identification ball (1), the anchoring filament (2) and the flange (6). Although in this case, in substitution of the interconnection filament (4) and the distal and proximal vertices of the first and second barriers respectively, is a single central obstructive body or “double single-plug”, comprising two elongated-barrel shaped barriers with size and configuration similar to (3 and 5), with a perimeter cleft (4) in the join zone of the two barriers configuring the model “B” or “double single-plug” device.

In order to facilitate the infra-vans deferens insertion and implantation of the two models “A” and “B” of the male contraception device as shown by FIGS. 1 and 2 on the drawing, the same insertion needle model is used for both, FIG. 3 (male option), producing complete assembly between the final third of the “bare” contraceptive device flange, Figures (1 and 2) and the “dressed” or “assembled” version, with its insertion needle fitted with a pivot (1) with washers (2 and 3) to which the flange tip (8) is axially connected in both model “A” and “B” of the improved device by sealing with injected liquid silicone, previously programmed in the automated occlusive contraception device manufacturing process, for which, at the moment, only shows two versions of the invention, with which to achieve an ideal contraceptive device, in other words, well assembled, solid and indicated for simple intra-duct implantation of both models of the cited invention.

It should be emphasised that the insertion and implantation needles for the occlusive intra-vas deferens device are disposable and made of a mixture of polyamide and fibre glass, although another polymer or stainless steel could also be used. On the other hand, models “A” and “B” of the male contraception device are made from medical grade silicone, although another fully biocompatible, inert polymer that is non-deformable, semi-elastic and with a minimum lifetime of one hundred years could be employed.

Lastly, it is not considered necessary to extend this description any further for those skilled in the art to understand the scope of the invention and its associated advantages. The materials, shape, size and layout of the elements making up the device are subject to variation, provided this does not involve any alterations to the essence of the invention.

1. OCCLUSIVE INTRA-VAS DEFERENS DEVICE for obtaining male sterility comprising an axial filament, with two occlusive elongated barrel-shaped bodies.

2. OCCLUSIVE INTRA-VAS DEFERENS DEVICE for obtaining male sterility in accordance with claim 1, further comprising a flange at one end of the axial filament which is adapted to be joined to a surgical needle.

3. OCCLUSIVE INTRA-VAS DEFERENS DEVICE for obtaining male sterility in accordance with claim 1, further comprising a ball at an opposite end of the axial filament.

4. OCCLUSIVE INTRA-VAS DEFERENS DEVICE for obtaining male sterility in accordance with claim 2, further comprising a needle assembled onto the flange that protrudes from the one end of the most peripheral elongated barrel-shaped body.
